(19) United States
(12) Patent Application Publication
(10) Pub. No.: US 2015/0344915 A1
(43) Pub. Date: Dec. 3, 2015

(54) PROCESS AND APPARATUS FOR PRODUSING CHEMICAL PRODUCT

(71) Applicant: Asahi Glass Company, Limited, Tokyo (JP)

(72) Inventors: Takashi SEKIYA, Tokyo (JP); Hiroshi HATANO, Tokyo (JP); Hiroki TANAKA, Tokyo (JP); Nobuyuki KASAHARA, Tokyo (JP)

(73) Assignee: Asahi Glass Company, Limited, Tokyo (JP)

(21) Appl. No.: 14/821,932

(22) Filed: Aug. 10, 2015

Related U.S. Application Data
(63) Continuation of application No. PCT/JP2014/057873, filed on Mar. 20, 2014.

(30) Foreign Application Priority Data
Mar. 28, 2013 (JP) 2013-070323

(51) Int. Cl. C12P 7/56; C12M 1/00

(52) U.S. Cl. CPC C12P 7/56; C12M 29/00; C12M 47/02; C12M 29/18

(57) ABSTRACT

A process for producing a chemical product comprises a first fermentation step wherein fermentation is conducted by supplying a starting material compound and oxygen to a microorganism-containing liquid, to obtain a first fermentation broth containing a chemical product formed by the fermentation, a second fermentation step wherein the first fermentation broth is taken out and used as a second fermentation broth, and fermentation is conducted by supplying oxygen without supplying a starting material compound, so that the concentration of the starting material compound in the second fermentation broth is adjusted to be a concentration (Y) lower than a concentration (X) of the starting material compound in the first fermentation broth, and a separation step wherein the second fermentation broth is separated to obtain a separated liquid containing the chemical product.

### TECHNICAL FIELD

The present invention relates to a process and apparatus for producing a chemical product from a starting material compound by fermentation.

### BACKGROUND ART

Methods for producing various chemical products via fermentation steps by means of microorganisms have been proposed. For example, the following Patent Document 1 discloses a method for producing lactic acid from sugar by fermentation employing a specific fission yeast.

Examples in the following Patent Document 2 disclose a method for continuously producing lactic acid by such a process that fermentation is conducted by supplying a microorganism and a culture medium (starting material sugar and ammonium sulfate) to a fermenter to produce lactic acid, a fermentation broth taken out from the fermenter is subjected to membrane separation to separate lactic acid and the microorganism, and the microorganism is returned to the fermenter.

### PRIOR ART DOCUMENTS

### Patent Documents

Patent Document 1: WO2011/021629

Patent Document 2: WO2012/077742

### DISCLOSURE OF INVENTION

### Technical Problem

However, in the method disclosed in Patent Document 2, the starting material sugar is present at a certain concentration in the fermentation broth in the fermenter, and in a permeated liquid (separated liquid) obtainable by membrane separation of the fermentation broth, not only the chemical product, but also the starting material sugar is contained, whereby a purification step is further required in order to separate the chemical product and the starting material sugar in the permeated liquid. The larger the amount of the starting material sugar contained in the permeated liquid, the lower the utilization efficiency of the starting material sugar, and the heavier the load in the purification step.

The present invention has been made in view of the above situations, and it is an object of the present invention to provide, in a method for producing a chemical product from a starting material compound by fermentation, a process for producing the chemical product whereby the amount of the starting material compound contained in the separated liquid can be reduced, and a production apparatus to be used for such a process.

### Solution to Problem

The present invention provides the following [1] to [8].

[1] A process for producing a chemical product, comprising:

a first fermentation step wherein fermentation is conducted by supplying a starting material compound and oxygen to a microorganism-containing liquid, to obtain a first fermentation broth containing a chemical product formed by the fermentation,

a second fermentation step wherein the first fermentation broth is taken out and used as a second fermentation broth, and fermentation is conducted by supplying oxygen to the second fermentation broth without supplying a starting material compound, so that the concentration of the starting material compound in the second fermentation broth is adjusted to be a concentration (Y) lower than a concentration (X) of the starting material compound in the first fermentation broth, and

a separation step wherein the second fermentation broth in which the concentration of the starting material compound is the concentration (Y), is taken out and used as a third fermentation broth, and the third fermentation broth is separated into a separated liquid containing the chemical product and not containing the microorganism, and a non-separated liquid containing the microorganism, to obtain the separated liquid containing the chemical product.

[2] The process for producing a chemical product according to [1], which further has a liquid returning step wherein the non-separated liquid containing the microorganism, obtained in the separation step, is supplied to the first fermentation step.
[3] The process for producing a chemical product according to [1] or [2], wherein the concentration (X) of the starting material compound in the first fermentation broth is from 5 to 50 g/L, and the concentration (Y) of the starting material compound in the second fermentation broth is at most 80% of the concentration (X).
[4] The process for producing a chemical product according to any one of [1] to [3], wherein the dissolved oxygen concentration in the first fermentation broth is from 10 to 300 ppb, and the dissolved oxygen concentration in the second fermentation broth is from 10 to 6,000 ppb.
[5] An apparatus for producing a chemical product, comprising:

a first fermentation part having a means of supplying a starting material compound to a microorganism-containing liquid, and a means of supplying oxygen to the microorganism-containing liquid, wherein a first fermentation broth containing a chemical product formed by fermentation, is obtained,

a separation part having a separation unit, wherein a separated liquid containing the chemical product and not containing the microorganism and a non-separated liquid containing the microorganism are obtained by separation,

a second fermentation part provided between the first fermentation part and the separation part, so that the first fermentation broth is taken out from the first fermentation part and used as a second fermentation broth, and having a flow path to send the second fermentation broth to the separation part, and a means of supplying oxygen to the second fermentation broth, wherein fermentation is conducted without supplying the starting material compound to the second fermentation broth, so that the concentration of the starting material compound in the second fermentation broth is adjusted to be a concentration (Y) lower than a concentration (X) of the starting material compound in the first fermentation broth.

[6] The apparatus for producing a chemical product according to [5], wherein the first fermentation part has a first fermenter, and the second fermentation part has a second fermenter.
[7] The apparatus for producing a chemical product according to [5] or [6], wherein the separation part has the separation unit and a recycling path to supply the non-separated liquid of the separation unit again to the separation unit.
[8] The apparatus for producing a chemical product according to any one of [5] to [7], which further has a liquid returning part having a flow path to send the non-separated liquid containing the microorganism from the separation part to the first fermentation part.

### Advantageous Effects of Invention

According to the present invention, in a process for producing a chemical product from a starting material compound by fermentation, at the time of obtaining a separated liquid containing the chemical product by separating the fermentation broth, it is possible to reduce the amount of the starting material compound contained in the separated liquid. It is thereby possible to improve the utilization efficiency of the starting material compound. Further, the amount of the starting material compound which should be removed at the time of purifying the separated liquid, is reduced, whereby the load in the purification step will be reduced.

### BRIEF DESCRIPTION OF DRAWINGS

FIG. 1 is a schematic configuration diagram illustrating an embodiment of the apparatus for producing a chemical product of the present invention.

FIG. 2 is a schematic configuration diagram illustrating an embodiment of the apparatus for producing a chemical product of the present invention.

### DESCRIPTION OF EMBODIMENTS

### Apparatus for Producing Chemical Product

The apparatus for producing a chemical product of the present invention comprises a first fermentation part, a second fermentation part, and a separation part. It is preferred that the apparatus for producing a chemical product of the present invention further has a liquid returning part.

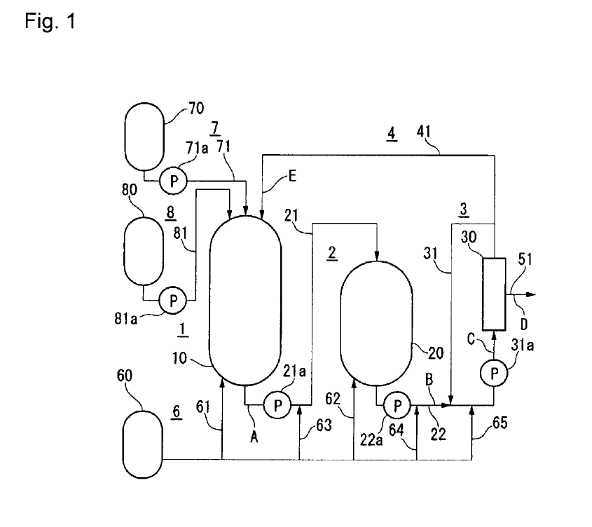

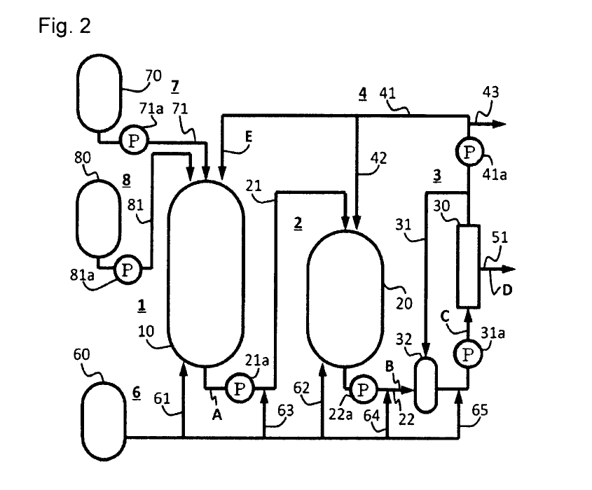

FIG. 1 and FIG. 2 are schematic configuration diagrams each illustrating a preferred embodiment of the apparatus for producing a chemical product, which is useful for carrying out the process for producing a chemical product of the present invention. The following description of the production apparatus will be made primarily with reference to FIG. 1 (in some cases with reference to FIG. 2).

The apparatus for producing a chemical product of this embodiment generally comprises a first fermentation part 1 having a first fermenter 10, a second fermentation part 2 having a second fermenter 20, a separation part 3 having a separation unit 30, and a liquid returning part 4 to send a liquid from the separation part 3 to the first fermentation part 1.

In the apparatus in this embodiment, a recycling system is formed such that a fermentation broth obtained in the first fermenter 10 is, after via the second fermenter 20, separated in the separation part 3, and a non-separated liquid containing the microorganism is returned, via the liquid returning part 4, to the first fermenter.

Here, in this specification, fermentation means treatment to convert a starting material compound by means of a microorganism to obtain a desired chemical product. In this specification, a fermentation broth means a liquid subjected to fermentation and contains the microorganism and the chemical product formed by the fermentation. Further, the fermentation broth may contain the starting material compound.

The first fermentation broth means a liquid containing the microorganism and the chemical product, present inside of the first fermentation part 1. Whereas, the second fermentation broth means a liquid containing the microorganism and the chemical product, present inside of the second fermentation part 2, after taken out from the first fermentation part 1 and till sent to the separation part 3. Further, the third fermentation broth means a liquid containing the microorganism and the chemical product, present inside of the separation part 3.

### [First Fermentation Part]

The first fermentation part in the present invention, has a means of supplying a starting material compound to a microorganism-containing liquid and a means of supplying oxygen to the microorganism-containing liquid, to obtain a first fermentation broth containing a chemical product formed by fermentation. The first fermentation part preferably comprises a first fermenter. The microorganism-containing liquid may be one containing at least a microorganism and may contain, in addition to the microorganism, a chemical product formed by fermentation. Further, it may contain, in addition to the microorganism, a starting material compound.

In the embodiment shown in FIG. 1, the first fermentation part 1 comprises a first fermenter 10. The first fermenter 10 is provided with a starting material supply means 7 to supply a starting material compound into the fermenter, a microorganism supply means 8 to supply a microorganism into the fermenter, and an oxygen supply means 6 to supply oxygen into the fermenter.

In this embodiment, the oxygen supply means 6 is designed so that it can supply oxygen also to the second fermentation part 2 and the separation part 3, respectively. That is, the oxygen supply means 6 serves also as an oxygen supply means for the second fermentation part and an oxygen supply means for the separation part.

Further, although not shown in the FIG., the first fermenter 10 is provided with a mixing means to uniformly mixing the interior of the fermenter, a gas discharge means to discharge an excess gas from the fermenter, and a temperature-adjusting means to maintain the liquid temperature in the fermenter at a prescribed temperature.

Further, the first fermenter 10 is, although not shown in the FIG., provided with devices to monitor the oxygen concentration, the starting material compound concentration and the microorganism concentration in the liquid in the fermenter. Control means are provided to control the starting material supply means 7, the microorganism supply means 8 and the oxygen supply means 6, so that the values obtainable from the monitor devices would be maintained to be constant.

The material and shape of the first fermenter 10 are not particularly limited, and a known fermenter may suitably be employed. In the present invention, oxygen is introduced into the liquid, such being considered to be an environment where corrosion of a metal is likely to take place relatively easily. Therefore, as the material for the device, it is preferred to employ glass or corrosion-resistant steel. Especially in a case where the desired chemical product exhibits acidity in the liquid, it is particularly preferred to employ glass or corrosion-resistant steel. As such glass, whole or part of the device may be made of glass, or a glass-lined steel may be used. As such corrosion-resistant steel, it is preferred to use stainless steel or a nickel alloy. Further, with respect to the material, it is preferred to employ the same material for the entire apparatus of the present invention. However, in a case where a membrane separation unit is adopted for the separation part, the material for the membrane is as will be described later. Further, it is preferred that the first fermenter 10 can be hermetically closed, and the inside can be maintained in a prescribed pressure state in order to prevent germs from entering from outside.

As the first fermenter 10 in this embodiment, a bubble-column fermenter, a stirrer-equipped fermenter or a tubular fermenter may, for example, be suitably used.

The capacity of the first fermenter 10 is not particularly limited and may suitably be set. In this embodiment, the capacity of the first fermenter 10 is preferably at least 0.3 L, more preferably at least 100 L, further preferably at least 1 m³, from such a viewpoint that the effects by the construction of this embodiment can thereby be readily obtainable and from the viewpoint of the production efficiency for the chemical product. The upper limit for the capacity is preferably at most 1,000 m³, more preferably at most 600 m³, whereby periodic maintenance check is easy.

The starting material supply means 7 comprises, for example, a starting material tank 70 to store a liquid containing a starting material compound (hereinafter referred to as a starting material-containing liquid), a starting material-containing liquid supply line 71 to send the starting material-containing liquid from the starting material tank 70 to the first fermenter 10, a pump 71a to send the starting material-containing liquid from the starting material tank 70 to the first fermenter 10, and a control means (not shown) to control the supply amount by adjusting the pump 71a. The starting material-containing liquid is, while being controlled, continuously or intermittently supplied to the first fermenter 10. Here, only one starting material tank 70 may be provided, or a plurality of such tanks may be provided.

The method for adjusting the pump 71a may, for example, be a method of directly controlling the driving power (electricity or frequency) for the pump, a method of controlling opening degrees of valves provided before and after the pump, a method of controlling the flow rate in a circulating line, by providing the circulating line to return the liquid from the discharge side to the inlet side of the pump, or a method of a combination thereof. The same applies to the method for adjusting pumps 81a, 21a, 22a, 31a and 41a which will be described later.

The microorganism supply means 8 comprises, for example, a cultivation tank 80 wherein a microorganism is cultivated to obtain a culture broth (liquid containing the microorganism) and the culture broth is stored, a culture broth supply line 81 to send the culture broth from the cultivation tank 80 to the first fermenter 10, a pump 81a to send the culture broth from the cultivation tank 80 to the first fermenter 10, and a control means (not shown) to control the supply amount by adjusting the pump 81a. The culture broth is, while being controlled, continuously or intermittently supplied to the first fermenter 10.

To the cultivation tank 80, a liquid culture medium and a microorganism are supplied, and a gas containing oxygen is supplied, and the tank is maintained at a prescribed culturing temperature. By such operations, the microorganism is cultivated to obtain a culture broth having a prescribed microorganism concentration. Depending upon the type of the microorganism, a known culture medium and culturing conditions may be employed.

In the culture medium, the starting material compound may be contained. In such a case, when the culture broth is supplied to the fermenter by the microorganism supply means 8, the microorganism and the starting material compound are simultaneously supplied.

The oxygen supply means 6 comprises, for example, a gas storage tank 60 to store a gas containing oxygen, as pressurized, a gas supply line 61 to send the gas from the gas storage tank 60 to the first fermenter 10, and a control means (not shown) to control the supply amount by adjusting a valve not shown. Oxygen is, while being controlled, continuously or intermittently supplied to the first fermenter 10. Oxygen is usually supplied in the form of a gas. The gas to be supplied may be any gas so long as it contains oxygen and is a gas which presents no adverse effect to fermentation. For example, it may be pure oxygen, a mixed gas of oxygen and at least one type of gas other than oxygen (such as air, nitrogen, carbon dioxide or methane), or air. From the viewpoint of availability, it is preferred to use air.

The oxygen concentration of the gas to be supplied into the tank of the first fermenter 10 is preferably from 5 to 50 vol %, more preferably from 15 to 30 vol %. When the oxygen concentration is at least the lower limit value in the above range, it becomes easy to supply a sufficient amount of oxygen to be utilized by the microorganism. Further, when the oxygen concentration is at most the upper limit value in the above range, the load to increase the oxygen concentration decreases, whereby it becomes easy to supply the gas.

The oxygen supply means 6 preferably has such a construction that the gas is supplied from a lower portion of the first fermenter 10 so that the liquid in the fermenter is stirred. That is, the first fermenter 10 is preferably a bubble column fermenter. Further, a construction having a draft tube provided inside is preferred from the viewpoint of good stirring efficiency. Such a construction is preferred in that the structure of a large sized fermenter can be simplified, and a damage to the microorganism can easily be prevented.

The detailed structure to supply a gas into the fermenter may, for example, be a perforated-pipe distributor (sparger), a gas injection device, a gas permeation membrane type device, etc. The perforated-pipe distributor may, for example, be a tubular sparger having many perforations formed in a straight or ring-shaped tube, or a sintered metal sparger employing a sintered metal having many voids. The gas injection device may, for example, be a gas injection nozzle type injection device to inject a high pressure gas from a nozzle, a two-fluid nozzle type injection device to let a high pressure gas and a high pressure liquid be injected from the respective nozzles and collided, or an aspirator type injection device to aspirate a gas by a high speed liquid. Particularly in the case of the gas injection nozzle type injection device, by adjusting the nozzle shape, it is possible to make it a device to form fine gas bubbles (so-called micro-bubbles or nano-bubbles). As the gas permeable membrane type device, a device may be exemplified wherein a gas permeable membrane is used as a wall surface of the tank or as a part of e.g. a baffle plate for stirring, to let a gas be permeated through the permeable membrane and dissolved in a liquid. Such detailed structures may be used in combination.

Further, the first fermenter 10 preferably has a gas discharge means capable of discharging a gas collected at the upper portion of the fermenter, as the case requires. The discharged gas may be recovered and returned again into the system.

As the concentration monitor for oxygen in the liquid in the fermenter, a common dissolved oxygen meter may be employed. As the concentration monitors for the starting material compound and the desired chemical product, a near infrared sensor, an enzyme electrode, etc. may be employed. Otherwise, a test sample may be sampled and measured by means of e.g. high performance liquid chromatography (HPLC). As the concentration monitor for the microorganism, an optical sensor or a capacitance sensor may be employed.

### [Second Fermentation Part]

The second fermentation part in the present invention is provided between the first fermentation part and the separation part, so that the first fermentation broth is taken out from the first fermentation part and used as a second fermentation broth, and it has a flow path to send the second fermentation broth to the separation part, and a means of supplying oxygen to the second fermentation broth, wherein fermentation is conducted without supplying the starting material compound to the second fermentation broth, so that the concentration of the starting material compound in the second fermentation broth is adjusted to be a concentration (Y) lower than a concentration (X) of the starting material compound in the first fermentation broth. The second fermentation part preferably comprises a second fermenter.

In the embodiment shown in FIG. 1, the second fermentation part 2 comprises flow paths (pipings) 21, 22 to send a liquid from the first fermentation part 1 to the separation part 3, and a second fermenter 20 provided between the paths. In the FIG., reference symbol 21 represents a piping for connecting the second fermenter 20 and the first fermenter 10 on the first fermentation part side, which is provided with a pump 21a. In the FIG., reference symbol 22 represents a piping for connecting the second fermenter 20 and the after-mentioned recycling path 31 of the separation part 3 on the separation part side, which is provided with a pump 22a.

The second fermenter 20 is provided with an oxygen supply means 6 to supply oxygen into the fermenter. Further, although not shown in the FIG., the second fermenter 20 is provided with a mixing means to uniformly mix the interior of the fermenter, a gas discharge means to discharge an excess gas from the fermenter, and a temperature-adjusting means to maintain the liquid temperature in the fermenter at a prescribed temperature.

Further, the second fermenter 20 is, although not shown in the FIG., provided with devices to monitor the oxygen concentration, the starting material compound concentration and the microorganism concentration in the liquid in the fermenter. Control means to control pumps 21a and 22a provided, respectively, for the piping 21 on the first fermentation part side and for the piping 22 on the separation part side, and a control means to control the oxygen supply means 6, are provided, so that the values obtainable from the monitor devices would be maintained to be constant.

Other constructions commonly known in usual fermenters, such as pH controlling means, liquid level controlling means, etc. may suitably be provided.

The material and shape of the second fermenter 20 are not particularly limited, and a known fermenter may suitably be employed. The material for the device is the same as in the case of the first fermenter 10. Further, it is preferred that the second fermenter 20 can be hermetically closed, and the inside can be maintained in a prescribed pressure state in order to prevent germs from entering from outside.

As the second fermenter 20 in this embodiment, a bubble-column fermenter, a stirrer-equipped fermenter or a tubular fermenter may, for example, be suitably used. As the second fermenter 20, it is not necessarily required to have an independent shape as a tank. That is, it is simply required to have an oxygen supply means to supply oxygen, a means capable of discharging an excess gas and a construction whereby the retention time can be secured for the fermentation broth. For example, it may be of such a simple construction that oxygen can be supplied to a long pipe or a thick pipe and ventilation can be done from a gas pool. However, the second fermenter is preferably a tank having a prescribed capacity, since it is required to control the oxygen concentration and the temperature. Further, as the second fermenter 20, one vessel may be provided alone, or a plurality of vessels may be provided in series or in parallel. Particularly in a case where the first fermenter 10 and the second fermenter 20 are large-sized, and it takes time for sending the liquid, it is preferred that a plurality of vessels are provided in parallel. For example, it is preferred that three vessels are provided in parallel, to present such an apparatus construction that three steps of (1) a step of receiving the second fermentation broth having a high starting material compound concentration from the first fermenter, (2) a step of continuously supplying oxygen to lower the concentration of the starting material compound, and (3) a step of sending the second fermentation broth having the starting material compound concentration lowered, to the separation part, can be proceeded in parallel.

The capacity of the second fermenter 20 is not particularly limited and may suitably be set. In this embodiment, the capacity of the second fermenter 20 is preferably at least 0.3 L, more preferably at least 100 L, further preferably at least 1 m³, from such a viewpoint that the effects by the construction of this embodiment can thereby be readily obtainable and from the viewpoint of the production efficiency. The upper limit for the capacity is preferably at most 1,000 m³, more preferably at most 600 m³, whereby periodic maintenance check is easy. Further, the capacity (capacity ratio) of the second fermenter 20 to the first fermenter 10 is preferably from 0.01 to 2, more preferably from 0.05 to 1, when the capacity of the first fermenter is regarded to be 1. When the capacity ratio is at least the lower limit value in the above range, the concentration of the starting material compound in the separation part 3 can easily be lowered. Further, when the capacity ratio is at most the upper limit value in the above range, the apparatus efficiency can easily be made high.

In the second fermenter 20, the microorganism utilizes the starting material compound in the second fermentation broth, whereby the concentration of the starting material compound in the second fermentation broth decreases. By prolonging the average retention time (effective capacity/average volume flow rate) in the second fermenter 20, it is possible to lower the concentration of the starting material compound in the second fermentation broth. Here, the effective capacity is the sum of the effective capacity of the second fermenter 20 (the capacity actually filled with the liquid, and in a case where a plurality of second fermenters 20 are present, their total capacity) and the capacities of pipings 21 and 22. Whereas, the average volume flow rate is based on the amount of the liquid sent out from the first fermenter 10.

The first fermentation part side piping 21 and the separation part side piping 22 may be provided with temperature adjusting means (not sown) as the case requires, so that the liquid temperatures in the pipings can be maintained at the prescribed fermentation temperatures.

The oxygen supply means 6 comprises, for example, a gas storage tank 60, a gas supply line 62 to send the gas from the gas storage tank 60 to the second fermenter 20, and a control means (not shown) to control the supply amount by adjusting a valve not shown. Oxygen is, while being controlled, continuously or intermittently supplied to the second fermenter 20. Oxygen is usually supplied in the form of a gas. The gas to be supplied may be the same as one described above as supplied to the first fermenter 10.

The oxygen concentration of the gas to be supplied into the tank of the second fermenter 20 is preferably from 5 to 50 vol %, more preferably from 15 to 30 vol %. When the oxygen concentration is at least the lower limit value in the above range, it becomes easy to supply a sufficient amount of oxygen to be utilized by the microorganism. Further, when the oxygen concentration is at most the upper limit value in the above range, the load to increase the oxygen concentration decreases, whereby it becomes easy to supply the gas.

The oxygen supply means 6 preferably has such a construction that the gas is supplied from a lower portion of the second fermenter 20 so that the liquid in the fermenter is stirred. That is, the second fermenter 20 is preferably a bubble column fermenter. Further, a construction having a draft tube provided inside is preferred from the viewpoint of good stirring efficiency. Such a construction is preferred in that the structure of a large sized fermenter can be simplified, and a damage to the microorganism can easily be prevented.

The detailed structure to supply a gas into the fermenter may be the same as one described above in the case of the first fermenter 10.

Further, although not shown in the FIG., it is preferred to provide a means to monitor the oxygen concentration in the liquid in the first fermentation part side piping 21 and/or the separation part side piping 22, and, if required, a means to supply a gas containing oxygen into the first fermentation part side piping 21 and/or the separation part side piping 22. As the gas, the same one as described above as supplied to the first fermenter 10 may be used.

The oxygen concentration in the gas to be supplied into the first fermentation part side piping 21 and/or the separation part side piping 22 is preferably the same as the oxygen concentration of the gas to be supplied into the tank of the second fermenter 20.

In order to supply the gas into the first fermentation part side piping 21 and/or the separation part side piping 22, for example, a gas supply line 63 and/or 64 is employed. As its detailed structure, the same one as in the case of the first fermenter 10 (e.g. a perforated-pipe distributor (sparger), a gas injection device, a gas permeation membrane type device, etc.) may be exemplified.

Further, the second fermenter 20 preferably has a gas discharge means capable of discharging a gas collected at the upper portion of the fermenter, as the case requires. The discharged gas may be recovered and returned again into the system.

Further, as the concentration monitor for oxygen, the concentration monitors for the starting material compound and the desired chemical product, and the concentration monitor for the microorganism, respectively, the same ones as mentioned above in the case of the first fermenter 10 may be employed.

### [Separation Part]

The separation part in the present invention has a separation unit to obtain a separated liquid and a non-separated liquid by separation. The separated liquid contains the chemical product and does not contain the microorganism. Here, “does not contain the microorganism” means “does not substantially contain”, but the microorganism (viable microorganism) in a wet weight amount of at most 20 g/L (preferably at most 10 g/L) may be contained. The non-separated liquid contains the chemical product and contains the microorganism. The separation part is preferably provided with a recycling path to take out the liquid containing the microorganism from the separation unit and supply it again to the separation unit.

In the embodiment shown in FIG. 1, the separation part 3 comprises a separation unit 30 and a recycling path 31 to recycle the non-separated liquid not separated by separation in the separation unit 30 to the separation unit 30. To the recycling path 31, the separation part side piping 22 of the second fermenter 20 is connected, and a pump 31a is provided between the connected position and the separation unit 30. Further, it is preferred to provide a buffer tank 32 as shown in FIG. 2, at the connected position, whereby the operation of the pump 31a becomes easy.

The separation unit 30 may be a device capable of separating the obtained fermentation broth (the third fermentation broth: liquid containing the microorganism and the chemical product) into a liquid (separated liquid) containing the chemical product and not containing the microorganism, and a liquid (non-separated liquid) containing the microorganism, and for example, a membrane separation device, a centrifugal separation device, an extraction separation device, etc. may be employed. The separation unit 30 may be composed of only one unit, or a plurality of units may be provided in series or in parallel.

The membrane separation device may be one provided with a separation membrane to let the desired chemical product in the third fermentation broth pass therethrough and not to let the microorganism pass therethrough, and a known membrane separation device may suitably be employed. The separation membrane may be an organic membrane or an inorganic membrane. The material for the separation membrane may, for example, be polyvinylidene fluoride, polysulfone, polyether sulfone, polytetrafluoroethylene, polyethylene, polypropylene, ceramics, etc. Among them, polysulfone or polyether sulfone is preferred from such a viewpoint that it is relatively inexpensive, its durability is high, or it can be constantly supplied.

The shape of the separation membrane is not particularly limited, and, for example, a flat membrane, a hollow-fiber membrane, etc. may be mentioned.

The separation membrane is preferably a porous membrane having pores with an average pore size of from 0.01 to 3 μm, since the microorganism is less permeable, and the membrane has a relatively high permeation flux. The average pore size of the separation membrane is more preferably from 0.1 to 0.65 μm.

The treatment capacity (permeation flux) of the membrane separation device may vary depending upon the size of the device, but, for example, it is preferably from 1 to 100 L/m²/h, more preferably from 3 to 30 L/m²/h.

The centrifugal separation device may be any device so long as it is provided with a mechanism to centrifugally sediment the microorganism, and a screw decanter may, for example, be mentioned. The treatment capacity of the centrifugal separation device may be suitably selected from e.g. the capacity of the first fermenter 10.

The extraction separation device may be any device so long as it is capable of extracting the desired chemical product in the third fermentation broth, from the fermentation broth, by means of an extracting agent, and an extraction column, etc., may be exemplified. The extraction column may, for example, be a plate extraction column, a packed extraction column, etc. The type of extraction may, for example, be a counter-current extraction or a concurrent extraction. The extracting agent may, for example, be an alcohol, an ester, a ketone, an ether, an amine, etc., and in each case, it is preferred to use an organic compound having from about 5 to 40 carbon atoms.

The recycling path 31 may be provided with a temperature adjusting means (not shown) as the case requires, so that the liquid temperature in the pipe is maintained at a prescribed fermentation temperature.

The separation unit 30 is provided with a discharge pipe 51 to discharge the separated liquid. The discharge pipe 51 is provided with a pump (not shown).

Further, it is preferred to provide a means (not shown) to monitor the oxygen concentration in the liquid in the recycling path 31 and, as the case requires, an oxygen supply means 6 to supply a gas containing oxygen into the recycling path continuously or intermittently. The oxygen supply means 6 is preferably provided at least at one optional location of the recycling path 31. In order to supply the gas into the recycling path 31, for example, a gas supply line 65 may be used. As its detailed structure, the same one as mentioned above in the case of the first fermenter 10 may be exemplified.

As the gas, the same one as mentioned above as supplied to the first fermenter 10 may be employed. The oxygen concentration in the gas to be supplied to the recycling path 31 is preferably the same as the oxygen concentration in the gas to be supplied into the tank of the second fermenter 20.

### [Liquid Returning Part]

The liquid returning part in the present invention supplies the non-separated liquid containing the microorganism from the separation part to the first fermentation part.

In the embodiment shown in FIG. 1, the liquid returning part 4 comprises a piping 41 (flow path). The piping 41 connects the recycling path 31 of the separation part 3 and the first fermenter 10. In the embodiment shown in FIG. 2, the liquid returning part 4 further comprises a pump 41a, a piping 42 and a discharge pipe 43. The piping 42 is branched from the piping 41 and is connected to the second fermenter 20. The discharge pipe 43 discharges a part of the non-separated liquid continuously or intermittently. The connecting point of the piping 41 and the recycling path 31 is located between the connecting point of the recycling path 31 and the separation part side piping 22 of the fermentation part 2, and the outlet where the non-separated liquid is discharged from the separation unit 30. The piping 41 is preferably provided with a flow rate-controlling valve in the vicinity of the connecting point of the piping 41 and the recycling path 31. By such a control valve, it is possible to adjust the balance in the flow rate between the recycling path 31 and the piping 41. In a case where a plurality of separation parts 3 are provided, the liquid returning part 4 may be of such a construction that the liquids from the respective separation parts are put together and returned to the first fermentation part, or may be of such a construction that they are independently returned to the first fermentation part.

Further, although not shown in the FIG., it is preferred to provide a means to monitor the oxygen concentration in the liquid in the piping 41 and, as the case requires, an oxygen supply means 6 to supply a gas containing oxygen into the piping 41 continuously or intermittently. The oxygen supply means 6 is preferably provided at least at one optional location of the piping 41. In order to supply the gas into the piping 41, for example, a gas supply line (not shown) may be used. As its detailed structure, the same one as mentioned above in the case of the first fermenter 10 (e.g. a perforated-pipe distributor (sparger), a gas injection device, a gas permeation membrane type device, etc.) may be exemplified.

As the gas, the same one as mentioned above as supplied to the first fermenter 10 may be employed. The oxygen concentration in the gas to be supplied to the piping 41 is preferably the same as the oxygen concentration in the gas to be supplied into the tank of the second fermenter 20.

Here, the liquid returning part 4 is not necessarily required to return all amount of the liquid sent from the separation part 3 to the first fermentation part 1. Via the piping 42, a part may be returned to the second fermentation part 2, or all amount may be returned to the second fermentation part 2. Further, via the discharge pipe 43, a part may be discharged as a waste liquid.

### <Process for Producing Chemical Product>

The process for producing a chemical product of the present invention is a process for producing a chemical product from a starting material compound by fermentation by means of a microorganism.

### [Microorganism]

The microorganism in the present invention is an organism which has an ability to consume the starting material compound and produce a desired chemical product. The microorganism may be one occurring naturally, or one having its nature partially modified by mutation or genetic recombination. A conventional one known in fermentation may suitably be used.

Examples of the microorganism may be a yeast, an Escherichia coli, a lactic acid bacterium, a filamentous bacterium, Actinomycetes, etc.

Among them, a yeast is preferred since it is excellent in the productivity for a chemical product, and chemical resistance (against an alcohol or an acid). The yeast may, for example, be a budding yeast or a fission yeast. The budding yeast may, for example, be Kluyveromyces lactis, Torulaspora delbrueckii, Zygosaccharomyces bailii, Pichia pastoris, etc. The fission yeast may, for example, be Schizosaccharomyces pombe, Schizosaccharomyces japonicus, Schizosaccharomyces octosporus, etc.

Among the above fission yeasts, Schizosaccharomyces pombe (hereinafter referred to also as S. pombe) is preferred, in that various useful mutant strains can be utilized.

### [Starting Material Compound]

The starting material compound in the present invention is a compound which may be directly utilized by the microorganism, so that the desired chemical product is obtainable by its fermentation. A conventional one known in fermentation may suitably be employed.

Examples of the starting material compound may, for example, be a sugar (such as a monosaccharide (a pentose or a hexose), a disaccharide or a polysaccharide), an alcohol (such as glycerol), an amino acid (such as alanime, glycine or leucine), etc.

Among them, a sugar is preferred, in that it can easily be utilized as a carbon source by the microorganism. Preferred examples of the sugar may be a pentose such as ribose, arabinose or xylose; a hexose such as glucose, fructose or galactose; a disaccharide such as sucrose, trehalose, cellobiose or maltose; a polysaccharide such as cellulose or starch; etc. Among them, a hexose is preferred, and glucose is particularly preferred.

In a case where the microorganism is capable of utilizing only a monosaccharide, a disaccharide or polysaccharide may be used as preliminarily treated. For example, by mixing a diastatic enzyme to a starting material containing a disaccharide or polysaccharide in a starting material tank, a monosaccharide obtained by decomposition may be used. Further, a starting material (such as strained lees (molasses) of sugarcane or beet) containing a large amount of sugar such as glucose, may be directly used.

### [Starting Material-Containing Liquid]

The starting material-containing liquid is a liquid (usually an aqueous solution) containing the starting material compound. In addition to the starting material compound, it may contain metal elements such as K, Na, Mg, Ca, Fe, etc. minerals and vitamins. In the following embodiment, the starting material-containing liquid does not contain a microorganism.

### [Chemical Product]

The chemical product in the present invention is a chemical product which is formed by the microorganism in the fermentation broth. It may contain a chemical product as a byproduct in addition to the desired chemical product.

The chemical product may, for example, be an alcohol or an organic acid.

Examples of the alcohol may, for example, be ethanol, 2-propanol, 1,3-butanediol, 1,4-butanediol, propylene glycol, glycerol, etc.

Examples of the organic acid may, for example, be acetic acid, malonic acid, succinic acid, glycolic acid, lactic acid, malic acid, tartaric acid, citric acid, 3-hydroxypropionic acid, pyruvic acid, etc. Here, a hydroxycarboxylic acid is considered to be an organic acid.

Among them, an organic acid is preferred, and lactic acid, malic acid, succinic acid or 3-hydroxypropionic acid is particularly preferred, since high versatility and marketability (such as applications to synthetic fibers, vehicles and alternate plastics) are thereby expected.

The production process of the present invention is applicable also to a method of obtaining a chemical product by forming sedimentation of a neutralized salt, etc. However, the production process of the present invention is particularly suitable for a method of obtaining a chemical product in the form of an aqueous solution without forming sedimentation.

Further, the production process of the present invention is particularly suitable for a method for producing a chemical product, of which the boiling point is higher than water (100° C.). In the production process of the present invention, in a case where the separated liquid obtained by separating the microorganism is an aqueous solution containing a chemical product (a chemical product crude solution), it is conceivable to use distillation as a means to separate the obtained chemical product and water. However, usually, the starting material compound will be separated as a high boiling point component or residue in distillation. In such a case, if the boiling point of the desired chemical product is lower than water, separation by distillation is easy. On the other hand, if the boiling point of the desired chemical product is higher than water, it tends to be difficult to separate the desired chemical product and water. Therefore, by lowering the concentration of the starting material compound contained in the separated liquid (the chemical product crude solution), it is possible to reduce the load required for purification (particularly distillation purification) of the chemical product.

Now, an embodiment will be described wherein a chemical product is continuously produced by the production process of the present invention by using an apparatus having the construction of FIG. 1.

### [First Fermentation Step]

In the process for producing a chemical product in the present invention, in a first fermentation step, fermentation is conducted by supplying a starting material compound and oxygen to a microorganism-containing liquid, to obtain a first fermentation broth containing a chemical product formed by the fermentation.

In this embodiment, a liquid culture medium and a microorganism are preliminarily supplied to a cultivation tank 80, and while continuously supplying a gas containing oxygen, the temperature is maintained at a prescribed culturing temperature to obtain a culture broth. The oxygen concentration and the culturing temperature in the liquid (culture broth) in the cultivation tank 80 are controlled so that the cultivation conditions are maintained to be suitable for cultivation of the microorganism. Usually as between the cultivation conditions suitable for cultivation of the microorganism and the fermentation conditions suitable for the production of a chemical product by fermentation, preferred oxygen concentration conditions are different. Usually, the preferred oxygen concentration in the fermentation broth is lower than the oxygen concentration condition suitable for the cultivation.

Into the first fermenter 10, the starting material-containing liquid is supplied in a prescribed amount from the starting material supply means 7. The supply from the starting material supply means 7 may be conducted continuously or intermittently. Further, into the first fermenter 10, the culture broth containing the microorganism is supplied in a prescribed amount from the microorganism supply means 8. The supply from the microorganism supply means 8 may be conducted continuously or intermittently. Further, as described later, from the piping 41 of the liquid returning part 4, a liquid containing the microorganism (the non-separated liquid not separated in the separation unit) is supplied continuously or intermittently. In a case where the total (total supply amount) of the supply amount of the starting material-containing liquid from the starting material supply means 7, the supply amount of the culture broth containing the microorganism from the microorganism supply means 8 and the supply amount of the liquid containing the microorganism from the piping 41 of the liquid returning part 4, is at a constant rate, and the amount (discharge amount) of the fermentation broth sent out from the piping 21 is at a constant rate, and further, the rates of both are equal, the liquid surface level in the first fermenter 10 will be constant. On the other hand, the total supply amount and the discharge amount may not necessarily be made to be constant values, and these values may be up and down intermittently (intermittingly). For example, for a certain period of time, the total supply amount is set to be a certain value, and at the same time, the discharge amount is set to be zero, to let the liquid amount inside of the first fermenter increase. Then, both of the total supply amount and the discharge amount are set to be zero, and as the case requires, left for a certain period of time. Thereafter, while keeping the total supply amount to be zero, the discharge amount is set to be a certain value. Further, both of the total supply amount and the discharge amount are set to be zero, and as the case requires, left for a certain period of time. By repeating such an operation, the liquid surface level will undergo up and down. Such a quasi-batch system operation method may be employed.

While controlling the liquid temperature in the first fermenter 10 to be at a prescribed fermentation temperature, a gas containing oxygen is continuously supplied into the liquid by the oxygen supply means 6, and at the same time, the starting material compound is continuously or intermittently supplied from the starting material supply means 7. Fermentation thereby proceeds in the liquid, whereby the oxygen and the starting material compound are consumed, to form chemical products (the desired chemical product and chemical products formed as by-products).

The liquid in the first fermenter 10 is made to be substantially uniform by a stirring action as the gas is continuously supplied by the gas supply means 13. The concentration of the starting material compound in the first fermentation broth in the first fermenter 10 is designated as concentration (X). Here, in the second fermentation broth immediately after discharged from the first fermenter 10 to the first fermenter side piping 21 (at a location shown by symbol A in the FIG., hereinafter referred to as point A), the formed chemical products, the starting material compound, the microorganism and oxygen are contained in substantially the same concentrations as their concentrations in the first fermenter 10. That is, the concentration of the starting material compound in the second fermentation broth at point A is the same as the concentration (X) of the starting material compound in the first fermentation broth in the first fermenter 10. Therefore, by sampling the liquid at point A, the concentration (X) may be measured.

In a case where the amount of the microorganism (viable organism) in the liquid in the first fermenter 10 and the retention time in the first fermenter 10 are constant, the yield of the desired chemical product changes depending upon the oxygen concentration and the starting material compound in the liquid.

Therefore, the supply rate of oxygen and the supply rate of the starting material compound are controlled, and as the case requires, a culture medium containing the microorganism is supplied, so that the amount of the microorganism (viable organism), the oxygen concentration and the starting material compound in the liquid in the first fermenter 10 are maintained in such ranges wherein a good yield of the chemical product is obtainable.

In this specification, the yield is a yield against the starting material compound. The yield against the starting material compound is a value obtained by dividing the mass of the obtained chemical product by the mass of the consumed starting material compound. For example, in a case where 0.9 g of lactic acid is obtained by consumption of 1 g of glucose, the yield becomes 90%.

In this specification, the average retention time in a fermenter is a value obtained by dividing the effective capacity of the fermenter by the average volume flow rate. The effective capacity is the capacity actually filled with the liquid. Whereas, the average volume flow rate is the volume per unit time of the fermentation broth sent out from the fermenter. In the case of the first fermenter, in a continuous operation, the operation is conducted so that, per unit time, the total volume of liquids (the starting material liquid, the culture broth and the returned liquid) supplied to the fermenter, and the volume of the fermentation broth sent out from the fermenter become equal.

With respect the amount of the viable organism in the fermenter 10, a preferred range is determined by a preliminary fermentation test. That is, a preferred microorganism density of the viable organism is determined by the test and multiplied by the effective capacity of the fermenter 10 to obtain the amount of the viable organism. With respect to the microorganism density, although it may depend on the type of the microorganism and the culturing conditions, it is preferred to conduct fermentation at a high density to some extent in order to control the volume of the fermenter 10 to be small.

With respect the oxygen concentration in the fermenter 10, a preferred range is determined by a preliminary fermentation test. Particularly in the case of the present invention, it is essential to supply oxygen in fermentation. However, usually, if the oxygen concentration is increased, although the consumption rate of a starting material compound may be increased and the production rate of the desired chemical product may be increased, propagation of the microorganism tends to preferentially proceed. Therefore, it is preferred that the oxygen concentration in the fermenter 10 is controlled to be relatively low.

The average retention time in the fermenter 10 is calculated based on the fermentation rate. The fermentation rate is the consumption rate of the starting material compound per unit time per microorganism amount. With respect to the consumption rate of the starting material compound, a preferred range is determined by a preliminary fermentation test. In a case where the consumption rate is susceptible to an influence of the starting material compound concentration, a consumption rate is obtained in the desired starting material compound concentration range.

The starting material compound concentration in the fermenter 10 is set to be low to such an extent that the consumption rate will not thereby be extremely low. If the starting material compound concentration is set to be too low, the fermentation rate tends to decrease. On the other hand, if the starting material compound concentration is set to be too high, the utilization efficiency of the starting material compound tends to decrease.

By setting the respective values in consideration of the foregoing conditions, it is possible to increase the production rate for the desired chemical product. It is particularly preferred to increase the production rate of the chemical product per unit time per unit volume of the fermenter. However, individual elements to be controlled (such as the supply amount of the starting material compound, the supply amount of oxygen, the temperature, the pH and the discharge rate of the fermentation broth from the fermenter) are likely to interfere with one another, and therefore, the optimum values in the fermenter should finally be suitably adjusted by the actual operation.

For example, in the case of producing lactic acid as the desired chemical product by using yeast and glucose as the starting material compound, the amount of the viable organism (microorganism density) in the first fermenter 10 is preferably from 12 to 72 g/L, more preferably from 24 to 48 g/L, as calculated by dry weight. When the amount of the viable organism is at least the lower limit value in the above range, it is possible to increase the production rate of the chemical product per unit volume of the fermenter. Further, when it is at most the upper limit value, such is preferred in that the stress exerted to the microorganism can be controlled to be low, or it is possible to readily distribute oxygen and the starting material compound sufficiently and uniformly to the microorganism.

Further, the microorganism concentration (hereinafter referred to as “microorganism concentration OD660”) shown in Examples given hereinafter, etc., is a value calculated from the absorbance of light with a wavelength of 660 nm (OD660) measured by means of visible ultraviolet spectroscope V550 manufactured by JASCO Corporation. OD=1 at 660 nm corresponds to 0.2 g/L of the dry weight of yeast or 0.8 g/L of the wet weight of yeast.

The oxygen concentration in the liquid i.e. the dissolved oxygen concentration, in the first fermenter 10 is preferably from 10 to 300 ppb, more preferably from 20 to 150 ppb. When the dissolved oxygen concentration is at least the lower limit value in the above range, it is possible to prevent a decrease in the production rate for the chemical product, and when it is at most the upper limit value in the above range, it is possible to prevent a decrease in the yield, such being desirable.

The concentration (X) of the starting material compound in the liquid in the first fermenter 10 is preferably from 5 to 500 g/L, more preferably from 10 to 200 g/L. It is preferred that the concentration of the starting material compound is at least the lower limit value in the above range, in that a decrease in the production efficiency of the chemical product (a decrease in the consumption rate of the starting material compound by the microorganism) can easily be prevented, and the concentration of the obtainable chemical product can easily be increased. It is preferred that the concentration of the starting material compound is at most the upper limit value, in that the microorganism density of viable organism can easily be maintained at a high level, and the interior of the fermenter can easily be uniformly stirred.

The average retention time in the first fermenter 10 is preferably from 0.1 to 120 hours, more preferably from 1 to 60 hours.

The concentration of the desired chemical product in the liquid in the first fermenter 10 is preferably from 5 to 200 g/L, more preferably from 10 to 150 g/L. It is preferred that the concentration of the desired product is at least the lower limit value in the above range, in that the purification cost for the chemical product can easily be controlled, and it is preferred that the concentration is at most the upper limit value, in that a decrease in the production efficiency for the chemical product can easily be prevented.

The pressure in the first fermenter 10 (the pressure of the gas phase, the differential pressure from the atmospheric pressure) is not particularly limited, and is preferably at least normal pressure (atmospheric pressure) and at most 100 kPa.

### [Second Fermentation Step]

In the process for producing a chemical product in the present invention, in the second fermentation step, the first fermentation broth is taken out and used as a second fermentation broth, and fermentation is conducted by supplying oxygen to the second fermentation broth without supplying a starting material compound, so that the concentration of the starting material compound in the second fermentation broth is adjusted to be a concentration (Y) lower than a concentration (X) of the starting material compound in the first fermentation broth.

In this embodiment, the second fermentation broth discharged from the first fermenter 10 is, via the first fermentation part side piping 21, supplied to the second fermenter 20 continuously or intermittently and, after retained for a prescribed time in the second fermenter 20, via the separation part side piping 22, joined to a liquid flowing in the recycling path 31 of the separation part 3.

The liquid temperature in the second fermenter 20 is controlled at a prescribed fermentation temperature, and a gas containing oxygen is continuously supplied into the liquid, whereby fermentation proceeds in the liquid, and the starting material compound and oxygen are consumed to form chemical products (the desired chemical product and chemical products as by-products). The liquid in the second fermenter 20 is made to be substantially uniform by a stirring action as the gas is continuously supplied by the oxygen supply means 6.

In order to keep the microorganism alive in the second fermentation broth during passage of the broth through the first fermentation part side piping 21, a gas containing oxygen may be supplied to the broth in the first fermentation part side piping 21, as the case requires. Further, in this embodiment, it is so designed that the liquid temperature in the first fermentation part side piping 21 is maintained at a prescribed fermentation temperature. Therefore, also in the first fermentation part side piping 21, fermentation is continued, and the starting material compound and oxygen are consumed to form chemical products.

In order to keep the microorganism alive in the fermentation broth discharged from the second fermenter 20 during passage of the broth through the separation part side piping 22, a gas containing oxygen may be supplied to the broth in the separation part side piping 22, as the case requires. Further, in this embodiment, it is so designed that the liquid temperature in the separation part side piping 22 is maintained at a prescribed fermentation temperature. Therefore, in a case where the starting material compound remains in the fermentation broth discharged from the second fermenter 20, also in the separation part side piping 22, fermentation will be continued, and the starting material compound and oxygen contained in the fermentation broth will be consumed to form chemical products.

In this embodiment, the starting material compound contained in the second fermentation broth discharged from the first fermenter 10, will be consumed during the passage through the first fermentation part side piping 21, the second fermenter 20 and the separation part side piping 22. Accordingly, the concentration of the starting material compound in the second fermentation broth obtained in the second fermentation part 2 i.e. the second fermentation broth immediately before (location shown by symbol B in the FIG., hereinafter referred to as point B) introduced to the recycling path 31 of the separation part 3, is reduced to be lower than in the second fermentation broth discharged from the first fermenter 10.

In the present invention, concentration (Y) is the concentration of the starting material compound in the liquid taken out from the second fermentation part 2 and supplied to the separation part 3 as a third fermentation broth.

In this embodiment, the liquid in the second fermenter 20 is made to be substantially uniform by a stirring action as the gas is continuously supplied by the oxygen supply means 6. Further, in the second fermentation broth at point B, the formed chemical product and the starting material compound are contained in substantially the same concentrations as the concentrations in the second fermenter 20. That is, in this embodiment, the concentration of the starting material compound in the second fermentation broth in the second fermenter 20 and the concentration of the starting material compound in the second fermentation broth at point B are the same and concentration (Y).

And, by controlling the average retention time in the second fermentation part 2 i.e. the average retention time from immediately after discharged from the first fermenter 10 to immediately before introduced to the recycling path 31 of the separation part 3, it is possible to reduce the concentration (Y) of the starting material compound in the second fermentation broth to a desired level.

The average retention time in the second fermentation part 2 is the total of the transit time in the first fermentation part side piping 21, the average retention time in the second fermenter 20 and the transit time in the separation part side piping 22.

Preferably, a method of controlling the concentration (Y) of the starting material compound in the second fermentation broth by adjusting the average retention time in the second fermenter 20 by bringing the flow rates in the first fermentation part side piping 21 and the separation part side piping 22 to be constant at the respective prescribed values, is preferred to avoid complication of the operation.

In this embodiment, the concentration of the starting material compound in the second fermentation broth at point B (i.e. concentration Y) is preferably at most 80%, more preferably at most 50%, to the concentration (X) of the starting material compound in the first fermentation broth i.e. the concentration of the starting material compound in the second fermentation broth at point A (i.e. concentration X) immediately after discharged from the first fermenter 10 to the first fermentation part side piping 21.

The concentration (Y) of the starting material compound at point B is preferably at most 10 g/L, more preferably at most 8 g/L, further preferably at most 5 g/L, particularly preferably at most 2 g/L, and from the viewpoint of the load for purification of the chemical product, it is ideally zero.

When the concentration of the starting material compound in the second fermentation broth at point B is at most the upper limit value in the above range, it is possible to sufficiently lower the concentration of the starting material compound in the third fermentation broth immediately before (location shown by symbol C in the FIG., hereinafter referred to as point C) introduced to the separation unit 30 of the separation part 3. It is thereby possible to suitably reduce the amount of the starting material compound contained in the separated liquid of the separation part 3.

In a case where an aerobic fermentation (fermentation requiring oxygen) is conducted in this embodiment, the time to consume oxygen at the concentration in the first fermentation broth tends to be shorter than the time to consume the starting material compound at the concentration in the first fermentation broth. Therefore, by providing the second fermentation part, oxygen is supplied without supplying the starting material compound, in order to promote fermentation thereby to promote consumption of the starting material compound. By such a method, the utilization efficiency of the starting material compound will be made high, and the yield of the desired chemical product will be improved. At the same time, it is possible to lower the concentration of the starting material compound in the obtained crude liquid (separated liquid) of the chemical product and to reduce the load for purification of the chemical product.

With respect to the oxygen concentration in the liquid in the second fermenter 20 i.e. the dissolved oxygen concentration, a preferred range is obtained by a preliminary fermentation test. The lower limit for the dissolved oxygen concentration in the liquid in the second fermenter 20 is set so that the fermentation rate in the second fermenter 20 will not be extremely slow. On the other hand, the upper limit may basically be the saturated oxygen concentration. The purpose is to consume the starting material compound thereby to lower the concentration of the starting material compound in the separation part 3. However, in consideration of also the production efficiency of the desired chemical product, the dissolved oxygen concentration in the liquid in the second fermenter 20 is preferably in the same range as the dissolved oxygen concentration of the liquid in the first fermenter 10. The dissolved oxygen concentrations in the liquid in the first fermentation part side piping 21 and in the liquid in the separation part side piping 22 are preferably in the same range as the dissolved oxygen concentration in the liquid in the second fermenter 20.

The average retention time (having the same meaning as the reciprocal of the average volume flow rate) in the second fermentation part is set so as to lower the concentration of the starting material compound contained in the second fermentation broth to at most a prescribed concentration. If the average retention time is too short, it tends to be difficult to lower the concentration of the starting material compound. On the other hand, if the average retention time is extremely too long, the apparatus tends to be large-sized, such being undesirable.

The temperature in the second fermentation part 2 is preferably the same as or slightly higher than the temperature in the first fermenter 10. However, the temperature condition may vary depending upon the microorganism.

By setting the respective values in consideration of the foregoing conditions, it is possible to lower the concentration of the starting material compound and to increase the production rate for the desired chemical product. It is particularly preferred to increase the consumption rate of the starting material compound per unit time per unit volume of the fermenter. However, individual elements to be controlled (such as the supply amount of oxygen, the temperature, the pH and the discharge rate of the fermentation broth from the first fermenter) are likely to interfere with one another, and therefore, the optimum values in the fermenter should finally be suitably adjusted by the actual operation.

The dissolved oxygen concentration in the liquid in the second fermenter 20 is preferably from 10 to 6,000 ppb, more preferably from 20 to 500 ppb. The dissolved oxygen concentration is preferably at least the lower limit value in the above range, in that a decrease in the consumption rate of the starting material compound can thereby be prevented. The upper limit for the dissolved oxygen concentration is more preferably at most 500 ppb, further preferably at most 200 ppb, with a view to improving the yield of the desired chemical product.

The dissolved oxygen concentrations in the liquids in pipings 21 and 22 are the same as the dissolved oxygen concentration in the liquid in the second fermenter 20.

The average retention time in the second fermentation part 2 is preferably from 5 minutes to 20 hours, more preferably from 20 minutes to 5 hours. The average retention time in the second fermentation part 2 is preferably from 0.001 to 1, more preferably from 0.01 to 0.8, when the average retention time in the first fermenter 10 is regarded to be 1.

### [Separation Step]

In the separation part 3, the non-separated liquid not separated in the separation unit 30 is, via the recycling path 31, introduced again to the separation unit 30, and the second fermentation broth obtained in the second fermentation part 2 is permitted to join with the non-separated liquid flowing in the recycling path 31 and then supplied to the separation unit 30.

By providing such a recycling path 31, the flow rate of the liquid to be supplied to the separation unit 30 can be made larger than the flow rate in the separation part side piping 22 of the second fermentation part 2, whereby it is possible to increase the linear velocity of the liquid to be supplied to the separation unit 30 without changing the flow rate in the separation part side piping 22 of the second fermentation part 2. Especially, in a case where a membrane separation device is employed as the separation unit 30, it is possible to prevent clogging of the separation membrane, by increasing the linear velocity of the liquid flowing at the surface of the separation membrane.

In order to keep the microorganism alive in the liquid flowing in the recycling path 31, a gas containing oxygen is supplied to the liquid in the recycling path 31 (a piping to supply a gas containing oxygen to the liquid in the recycling path 31 is not shown in the FIG.). The locations and the number of oxygen supply means 6 to be installed may suitably be changed.

Further, in this embodiment, it is so designed that the liquid temperature in the recycling path 31 is maintained at a prescribed temperature. Therefore, in a case where the starting material compound remains in the second fermentation broth at point B, fermentation will be continued even in a flow path from the joint position of the separation part side piping 22 and the recycling path 31 to immediately before introduced to the separation unit 30, but since the flow rate in the recycling path 31 is large, the transit time in such a flow path is short, and fermentation here is at such a low level as negligible.

In this embodiment, the concentration of the starting material compound in the third fermentation broth at point C is preferably at most 8 g/L, more preferably at most 5 g/L, ideally zero.

The third fermentation broth at point C is a mixture of the second fermentation broth at point B and the non-separated liquid flowing in the recycling path 31. Accordingly, the concentration of the starting material compound in the third fermentation broth at point C can be controlled by the concentration of the starting material compound in the second fermentation broth at point B and the dilution rate when joined with the non-separated liquid flowing in the recycling path 31 (determined by the flow rate of the non-separated liquid and the flow rate of the second fermentation broth at point B).

In the separation unit 30, a separated liquid containing chemical products and not containing the microorganism, and a non-separated liquid containing the remaining chemical products and the microorganism, are obtained. The separated liquid is taken out via a discharge pipe 51. The concentration of the starting material compound in the separated liquid (location shown by symbol D in the FIG., hereinafter referred to as point D) discharged by the discharge pipe 51 is preferably at most 10 g/L, more preferably at most 8 g/L, further preferably at most 5 g/L, particularly preferably at most 2 g/L, ideally zero. The concentration of the desired chemical product is preferably from 10 to 200 g/L, more preferably from 50 to 150 g/L.

Further, the yield is preferably at least 40%, more preferably at least 80%.

With respect to the oxygen concentration in the separation unit 30 i.e. the dissolved oxygen concentration, a preferred range is determined by a preliminary fermentation test. The lower limit for the dissolved oxygen concentration in the liquid in the separation unit 30 is set so that the viable organism rate of the microorganism will not be extremely lowered. On the other hand, the upper limit may basically be the saturated oxygen concentration.

The ratio of the separated liquid to the non-separated liquid in the separation unit 30 depends on the performance of the separation unit. Especially in a case where a membrane separation device is employed as the separation unit 30, it is preferred to maintain the linear velocity at the surface of the membrane to be within a constant range, with a view to preventing clogging. The linear velocity at the membrane surface is determined by the balance of 1) the volume flow rate of the liquid received from the second fermentation part 2, 2) the volume flow rate of the liquid discharged as a separated liquid, 3) the volume flow rate of the liquid in the recycling path 31, and 4) the volume flow rate of the liquid sent out to the liquid returning part. Usually, the volume flow rate at point C is set to be larger to some extent than the volume flow rate at point B.

The dissolved oxygen concentration in the liquid in the separation unit 30 is preferably from 10 to 6,000 ppb, more preferably from 20 to 500 ppb.

In a case where a membrane separation device is used as the separation unit, the linear velocity at the membrane surface is preferably from 0.1 to 3 m/s, more preferably from 0.3 to 2 m/s.

### [Liquid Returning Step]

A part of the liquid flowing in the recycling path 31 of the separation part 3 is, via a piping 41 of a liquid returning part 4, supplied to the first fermenter 10 continuously or intermittently.

By providing such a liquid returning step, continuous fermentation becomes possible. That is, it becomes possible to continuously conduct a series of operations of supplying the starting material compound to the first fermenter 10, converting this starting material compound to the desired chemical product by fermentation and obtaining the desired chemical product in the separation part. The process for producing a chemical product of the present invention is effectively applicable also to a case where the fermentation is conducted in a batch system. However, the process for producing a chemical product of the present invention is very effective even in the case of continuous fermentation, as it is capable of increasing the utilization efficiency of the starting material compound constantly.

In order to keep the microorganism alive in the liquid flowing in the piping 41, a gas containing oxygen may be supplied as the case requires.

In this embodiment, the concentration of the microorganism in the liquid immediately before (location shown by symbol E in the FIG., hereinafter referred to as point E) introduced to the first fermenter 10 is preferably at least 80%, more preferably at least 90%, of the microorganism concentration in the fermentation broth at point A.

The oxygen concentration in the liquid in the piping 41 i.e. the dissolved oxygen concentration is the same as the dissolved oxygen concentration in the liquid in the separation unit 30.

Further, the volume flow rate in the piping 41 is determined from the balance of the volume flow rate of the liquid in the separation unit 30.

The dissolved oxygen concentration in the liquid in the piping 41 is preferably from 10 to 6,000 ppb, more preferably from 20 to 500 ppb.

In this embodiment, a part of the non-separated liquid may be discharged via a discharge pipe 43 as shown in FIG. 2. By conducting this discharge, a part of the microorganism is discharged from the production apparatus. With respect to the microorganism reduced in the first fermentation part, supplement is made by a microorganism supply means 8. By this operation, the microorganism to be used for fermentation will be withdrawn upon expiration of a certain time (average retention time). On the assumption that the microorganism will not proliferate so much in the first fermentation part or the second fermentation part, if the amount of the microorganism to be supplied from the microorganism supply means 8 is made equal to the amount of the microorganism to be discharged from the discharge pipe 41, the total amount of the microorganism present in both of the first fermentation part and the second fermentation part can be maintained to be substantially constant. Here, the average retention time of the microorganism can be calculated by dividing the total microorganism amount calculated from the total entire volume (the liquid amount at the time of actual operation) of the first fermentation part, the second fermentation part, the separation part and the liquid returning part, by the microorganism amount actually discharged per unit time. The average retention time of the microorganism is preferably from 100 to 2,000 hours, more preferably from 200 to 800 hours.

According to this embodiment, by maintaining the liquid temperature at the fermentation temperature without supplying the starting material compound while keeping the microorganism alive by supplying oxygen during the period until the second fermentation broth discharged from the first fermenter 10 reaches the separation unit 30, it is possible to let the starting material compound in the second fermentation broth be consumed.

Consequently, in the liquid at point C to be supplied to the separation unit 30, the concentration of the starting material compound is lowered than the liquid at point A, and the amount of the starting material compound contained in the separated liquid in the separation unit 30 is lowered.

Thus, it is possible to improve the utilization efficiency of the starting material compound supplied to the first fermenter. Further, the amount of the starting material compound to be removed at the time of purifying the permeate is reduced, whereby the load in the purification step is reduced.

### Examples

Now, the present invention will be described in detail with reference to Examples. However, it should be understood that the present invention is by no means restricted by the following description. In Examples, the unit “%” for a content means “mass %” unless otherwise specified.

### [Microorganism]

A fission yeast having a lactic acid fermentative ability was prepared by a method in Examples disclosed in the specification of WO2012/114979. That is, a transformant (ASP3054 strain) of fission yeast Schizosaccharomyces pombe having pyruvic acid decarboxylase gene (PDC2) chromosomally-depleted and having L-Lactate Dehydrogenase (L-LDH) of human origin chromosomally-integrated, was obtained. This ASP3054 strain was used as the microorganism in the following tests.

### [Culture Broth]

The microorganism was inoculated to 150 mL of YES culture medium (culture medium containing 0.5% of Difco yeast extract, 30 g/L of glucose and 50 mL/L of 20 times concentrated supplement and having pH adjusted to 4.5) and cultured. Then, using a 3 L glass vessel culture device manufactured by Komatsugawa Chemical Engineering Co., Ltd., inoculation was conducted to reduce the amount to 1/10, followed by culturing (by controlling the pH to be 3.9 and the dissolved oxygen concentration (hereinafter abbreviated as “DO”) to be 2 ppm). Here, as the culture medium, a semisynthetic culture medium (culture medium containing 20 g/L of Yeast Extract, 15 g/L of (NH₄)₂SO₄, 22 g/L of glucose, 8 g/L of KH₂PO₄, 5.34 g/L of MgSO₄7H₂O, 0.04 g/L of Na₂HPO₄, 0.2 g/L of CaCl₂.2H₂O, traces of metals and traces of vitamins and having pH adjusted to 4.5) was used, and as the supplemental culture medium to be gradually added, a culture medium (culture medium containing 50 g/L of Yeast Extract, 500 g/L of glucose, 9 g/L of KH₂PO₄, 4.45 g/L of MgSO₄.7H₂O, 3.5 g/L of K₂SO₄, 0.14 g/L of Na₂SO₄, 0.04 g/L of Na₂HPO₄, 0.2 g/L of CaCl₂.2H₂O, traces of metals and traces of vitamins and having pH adjusted to 4.5) was used. Finally, a yeast-containing liquid (culture broth) having a microorganism concentration OD660 of 180 (36 g/L calculated as dried weight of yeast) was obtained.

### [Starting Material Containing Liquid]

A liquid containing 87.4 g/L of glucose, 0.5% of Difco yeast extract, 2.2 g/L of Na₂HPO₄, 1.05 g/L of MgCl₂.6H₂O, 0.015 g/L of CaCl₂.2H₂O, 1.0 g/L of KCl, 0.04 g/L of Na₂SO₄, 3.0 g/L of potassium hydrogen phthalate, traces of metals, vitamins and biotin, was prepared and used as the starting material containing liquid.

### [Production Apparatus]

A production apparatus was prepared in accordance with the apparatus shown in FIG. 1. Two sets of 1 L glass vessel culture device manufactured by Komatsugawa Chemical Engineering Co., Ltd. were prepared and used as the first fermenter 10 and the second fermenter 20. Here, in order to supply a gas (air) to each fermenter, a pipe was inserted from above so that its end was located in the vicinity of the bottom side. That is, it was so arranged that the supply of the gas was conducted from the fermenter bottom into the liquid. For the supply of air, compressed air pressurized by an air compressor was used as filtered through a filter. Further, the fermenters were provided with stirrers for stirring the interior of the fermenters. As liquid-sending pumps (21a, 22a, 31a and 71a), cassette tube pumps (SMP-21, manufactured by Tokyo Rikakikai Co., Ltd.) were used. As the separation unit 30, a membrane separation device (average pore size: 0.2 μm, hollow fiber membrane made of polysulfone, Xapmpler CFP-2-E-3MA, manufactured by GE Healthcare, membrane area: 110 cm²) was used. For the measurement of DO, InPro6900 manufactured by Mettler-Toledo International Inc. was used. For the measurements of glucose, lactic acid and ethanol, enzyme electrode method bio-sensors BF-5 and BF-7 manufactured by Oji Scientific Instruments were used. Using these, the apparatus for producing a chemical product as shown in FIG. 1 was prepared.

### Example 1

Using glucose as the starting material compound, lactic acid was produced as the desired chemical product under the following conditions.

So that the liquid amount in the first fermenter 10 would be 500 mL and the liquid amount in the second fermenter would be 400 mL, culture broths were introduced into the respective fermenters. However, the liquid amount in the second fermenter includes volumes of the connecting tubes before and after the fermenter. The supply rate of the starting material liquid to the first fermenter 10 (the liquid sending rate of the pump 71a) and the liquid sending rate of the separated liquid discharged from the separation unit 30 were, respectively, adjusted to be 33 mL/hr. The liquid sending rate from the first fermenter 10 to the second fermenter 20 (the liquid sending rate of the pump 21a) and the liquid sending rate from the second fermenter 20 to the separation unit 30 (the liquid sending rate of the pump 22a) were, respectively, adjusted to be 100 mL/hr. That is, the average retention time in the first fermenter 10 was adjusted to be 5 hours, and the average retention time in the second fermenter 20 was adjusted to be 4 hours. Further, the liquid sending rate at the inlet of the separation unit 30 (the liquid sending rate of the pump 31a) was adjusted to be 300 mL/min. The linear velocity at the membrane surface on the primary side (the side where the microorganism was present) of the membrane was thereby 0.5 m/sec. Further, the permeation flow rate was 3 L/m²/hr.

The temperatures inside of the first fermenter 10 and the second fermenter 20 were adjusted to be 28° C. Further, the pressures inside of the first fermenter 10 and the second fermenter 20 were adjusted to be substantially normal pressures. The supply amount of air (oxygen concentration: 21 vol %, the same applies hereinafter) to the first fermenter 10 was adjusted to be 0.25 L/min., and the supply amount of air to the second fermenter 20 was adjusted to be 0.2 L/min. The rotational speed of the stirrer was adjusted to bring DO in the liquids inside of the first fermenter 10 and in the second fermenter 20 (i.e. the dissolved oxygen concentration in the first fermentation broth and the dissolved oxygen concentration in the second fermentation broth) to be from 70 to 100 ppb (aimed target: 80 ppb). Fluctuations in DO are considered to be attributable to that the consumption rate of glucose could not necessarily be made constant, since the supply of the starting material compound was intermittent. The glucose concentration inside of the first fermenter 10 became substantially constant upon expiration of 100 hours from the initiation of fermentation (the point of time when recycling of the liquid was initiated, was taken as zero). Further, at this point of time, the microorganism concentration OD660 inside of the first fermenter 10 and inside of the second fermenter 20 was 180. Under these conditions, a continuous operation was conducted for 1,000 hours. So that the microorganism concentration OD660 inside of the first fermenter 10 would be maintained at a level of 180, the fermentation broth was supplied to the first fermenter 10, as the case required. Further, at the same time, so as to control the total liquid amount to be constant, a part of the liquid sent from the separation unit 30 to the first fermenter 10 was branched and discharged.

Table 1 shows the glucose and lactic acid concentrations in the first fermentation broth (the liquid in the first fermenter 10), the glucose and lactic acid concentrations in the second fermentation broth (the liquid in the second fermenter 20), the glucose, lactic acid and ethanol concentrations in the separated liquid (the liquid at point D), and the yield of lactic acid in the separated liquid, upon expiration of 1,000 hours. Further, at the same timing, the fermentation broth inside of the fermenter 10 was sampled, and the viable organism ratio was obtained. The results are shown in Table 1. Here, the viable organism ratio was measured by the following method. Further, the measured values of the concentrations of the respective starting material compounds in the third fermentation broth at point C are not shown in Table 1, but in the apparatus used in this Example, they show the values equal to the glucose, lactic acid and ethanol concentrations in the separated liquid (the liquid at point D).

The fermentation broth was sampled in an amount of 10 μL and subjected to centrifugal separation (3,300 G, 10 minutes). To the precipitate after removing the supernatant, 10 μL of a Trypan Blue staining solution (TRYPAN BLUE 0.4% SOLUTION, manufactured by MP Biomedicals) was added. Microscopic observation was conducted, whereby the presence or absence of staining was confirmed with respect to a total number of about 300 microorganisms. White microorganisms were judged to be viable organisms, and blue microorganisms were judged to be dead organisms.

### Example 2

Lactic acid was produced in the same manner as in Example 1 except that the liquid amount in the first fermenter 10 was 600 mL, the average retention time was 6 hours, the supply amount of air to the first fermenter 10 was 0.3 L/min., and the supply amount of air to the second fermenter 20 was 0.15 L/min. The results are shown in Table 1.

### Example 3

Lactic acid was produced in the same manner as in Example 1 except that the supply amount of air to the second fermenter 20 was 1 L/min., and DO in the liquid in the second fermenter 20 was 4,000 ppb. The results are shown in Table 1.

### Comparative Example 1

Lactic acid was produced in the same manner as in Example 1 except that the second fermenter 20 and the gas supply lines 62, 63 and 64 were not provided, and the first fermenter and the recycling line of the separation unit 30 were connected via the pump 21a. The results are shown in Table 1.

### Comparative Example 2

Lactic acid was produced in the same manner as in Example 1 except that instead of supplying air in the second fermenter 20, nitrogen gas was supplied at a rate of 0.2 L/min. The results are shown in Table 1.

TABLE 1

| | Ex. 1 | Ex. 2 | Ex. 3 | Ex. 4 | Ex. 5 |
|---|---|---|---|---|---|
| Liquid amount in first fermenter | 500 | 600 | 500 | 500 | 500 |
| 10 (mL) | | | | | |
| Retention time in first fermenter | 5 | 6 | 5 | 5 | 5 |
| 10 (hr) | | | | | |
| Glucose concentration X in first | 18 | 15 | 15 | 18 | 38 |
| fermentation broth (g/L) | | | | | |
| Lactic acid concentration in first | 52 | 55 | 46 | 42 | 37 |
| fermentation broth (g/L) | | | | | |
| Liquid amount in second fermenter | 400 | 300 | 400 | — | 400 |
| 20 (mL) | | | | | |
| Retention time in second fermenter | 4 | 3 | 4 | — | 4 |
| 20 (hr) | | | | | |
| DO in second fermenter 20 (ppb) | 80 | 80 | 4000 | — | 5 |
| Glucose concentration Y in second | 6.0 | 6.0 | 3.0 | — | 36 |
| fermenter 20 (g/L) | | | | | |
| Lactic acid concentration in second | 60 | 61 | 52 | — | 39 |
| fermenter 20 (g/L) | | | | | |
| Glucose concentration in separated | 5.9 | 5.6 | 3.0 | 18 | 36 |
| liquid (g/L) | | | | | |
| Lactic acid concentration in | 60 | 62 | 52 | 42 | 40 |
| separated liquid (g/L) | | | | | |
| Yield in separated liquid (%) | 74 | 76 | 62 | 75 | 78 |
| Ethanol concentration in | 7.2 | 7.0 | 8.0 | 4.0 | 4.5 |
| separated liquid (g/L) | | | | | |
| Viable organism ratio (%) | 90 | 83 | 95 | 88 | 72 |

As shown by the results in Table 1, it was possible to effectively reduce the amount of the starting material compound contained in the permeated liquid of the separation unit 30 by conducting the fermentation by supplying air to the second fermenter 20.

### INDUSTRIAL APPLICABILITY

According to the present invention, at the time of obtaining a separated liquid containing a chemical product by separating a fermentation broth, it is possible to reduce the amount of a starting material compound contained in the separated liquid, whereby it is possible to improve the utilization efficiency of the starting material compound, and the amount of the starting material compound which should be removed at the time of purifying the separated liquid, is reduced, whereby it is possible to reduce the load in the purification step, such being useful in a process for producing a chemical product from a starting material compound by fermentation.

This application is a continuation of PCT Application No. PCT/JP2014/057873, filed on Mar. 23, 2014, which is based upon and claims the benefit of priority from Japanese Patent Application No. 2013-070323 filed on Mar. 28, 2013. The contents of those applications are incorporated herein by reference in their entireties.

### REFERENCE SYMBOLS

1: first fermentation part, 2: second fermentation part, 3: separation part, 4: liquid returning part, 6: oxygen supply means, 7: starting material supply means, 8: microorganism supply means, 10: first fermenter, 20: second fermenter, 21: first fermentation part side piping, 21a: pump, 22: separation part side piping, 22a: pump, 30: separation unit, 31: recycling path, 31a: pump, 32: buffer tank, 41, 42: pipings, 43: discharge pipe, 51: discharge pipe, 60: gas storage tank, 61, 62, 63, 64, 65: gas supply lines, 70: starting material tank, 71: starting material-containing liquid supply line, 71a: pump, 80: cultivation tank, 81: culture broth supply line, 81a: pump

What is claimed is:

1. A process for producing a chemical product, comprising:
a first fermentation step wherein fermentation is conducted by supplying a starting material compound and oxygen to a microorganism-containing liquid, to obtain a first fermentation broth containing a chemical product formed by the fermentation,
a second fermentation step wherein the first fermentation broth is taken out and used as a second fermentation broth, and fermentation is conducted by supplying oxygen to the second fermentation broth without supplying a starting material compound, so that the concentration of the starting material compound in the second fermentation broth is adjusted to be a concentration (Y) lower than a concentration (X) of the starting material compound in the first fermentation broth, and
a separation step wherein the second fermentation broth in which the concentration of the starting material compound is the concentration (Y), is taken out and used as a third fermentation broth, and the third fermentation broth is separated into a separated liquid containing the chemical product and not containing the microorganism, and a non-separated liquid containing the microorganism, to obtain the separated liquid containing the chemical product.

2. The process for producing a chemical product according to claim 1, which further has a liquid returning step wherein the non-separated liquid containing the microorganism, obtained in the separation step, is supplied to the first fermentation step.

3. The process for producing a chemical product according to claim 1, wherein the concentration (X) of the starting material compound in the first fermentation broth is from 5 to 50 g/L, and the concentration (Y) of the starting material compound in the second fermentation broth is at most 80% of the concentration (X).

4. The process for producing a chemical product according to claim 1, wherein the dissolved oxygen concentration in the first fermentation broth is from 10 to 300 ppb, and the dissolved oxygen concentration in the second fermentation broth is from 10 to 6,000 ppb.

5. An apparatus for producing a chemical product, comprising:
a first fermentation part having a means of supplying a starting material compound to a microorganism-containing liquid, and a means of supplying oxygen to the microorganism-containing liquid, wherein a first fermentation broth containing a chemical product formed by fermentation, is obtained,
a separation part having a separation unit, wherein a separated liquid containing the chemical product and not containing the microorganism and a non-separated liquid containing the microorganism are obtained by separation,
a second fermentation part provided between the first fermentation part and the separation part, so that the first fermentation broth is taken out from the first fermentation part and used as a second fermentation broth, and having a flow path to send the second fermentation broth to the separation part, and a means of supplying oxygen to the second fermentation broth, wherein fermentation is conducted without supplying the starting material compound to the second fermentation broth, so that the concentration of the starting material compound in the second fermentation broth is adjusted to be a concentration (Y) lower than a concentration (X) of the starting material compound in the first fermentation broth.

6. The apparatus for producing a chemical product according to claim 5, wherein the first fermentation part has a first fermenter, and the second fermentation part has a second fermenter.

7. The apparatus for producing a chemical product according to claim 5, wherein the separation part has the separation unit and a recycling path to supply the non-separated liquid of the separation unit again to the separation unit.

8. The apparatus for producing a chemical product according to claim 5, which further has a liquid returning part having a flow path to send the non-separated liquid containing the microorganism from the separation part to the first fermentation part.
